(19) United States
(12) Patent Application Publication
(10) Pub. No.: US 2009/0281474 A1
(43) Pub. Date: Nov. 12, 2009

(54) TAMPON PLEDGET HAVING IMPROVED EXPANSION AND PROCESS FOR PRODUCING SAME

(75) Inventors: Eugene Dougherty, JR., Camden-Wyoming, DE (US); Keith Edgett, Middletown, DE (US); Robert Jorgensen, Middletown, DE (US)

Correspondence Address: MICHAUD-DUFFY GROUP LLP, 306 INDUSTRIAL PARK ROAD, SUITE 206, MIDDLETOWN, CT, 06457, US

(73) Assignee: PLAYTEX PRODUCTS, INC., Westport, CT (US)

(21) Appl. No.: 12/116,246

(22) Filed: May 7, 2008

(51) Int. Cl. A61F 13/20

(52) U.S. Cl. 604/11; 604/367; 604/379; 604/385.18; 604/385.17; 28/120

(57) ABSTRACT

A feminine hygiene product is presented. The feminine hygiene product includes a body having a mass of compressed fibrous material, an insertion end and a withdrawal end. The body has an overall length L. The insertion end has a diameter A and the withdrawal end has a diameter B. In an initial state, diameter B is at least about equal to up to about ten percent greater than the diameter A. When exposed to a fluid, diameter B radially expands such that a ratio of diameter B to the diameter A is about 1.25 to 1. In one embodiment, the feminine hygiene product is a vaginal tampon and when exposed to a fluid, diameter B expands more rapidly than diameter A to substantially seal a bottom portion of a vaginal cavity and prevent bypass leakage.

### BACKGROUND OF THE INVENTION

1. Field of the Invention

This invention relates generally to absorbent catamenial tampon pledgets and methods for making such tampon pledgets. More particularly, the present invention relates to tampon pledgets having improved expansion and absorbency characteristics at a withdrawal end to improve leakage protection.

2. Description of the Related Art

The human female vagina is located in the lower pelvic cavity and is surrounded by organs such as the uterus, the bladder, and the rectum. The vagina is a collapsed tube-like structure composed of fibromuscular tissue layers that extends from the uterus inside the body to the vulva on the exterior of the body. The entrance to the vagina, the introitus, is of a relatively small diameter, a lower two-thirds of the vagina is only slightly larger in diameter (if at all) than the introitus, and an upper third (nearest the cervix of the uterus) is the widest diameter. From a biological perspective, the vagina provides a path for fluids to leave the body during the menstruation phase of the menstrual cycle. Imaging data (e.g., magnetic resonance imaging (MRI)) of the pelvic region of women during menstruation shows that women generally place tampon pledgets into one of the vaginal fornices, both anterior and to the right of the cervical os, the source of menstrual bleeding. Both because gravity tends to urge blood down the vaginal cavity toward the exterior of the body and the orientation of the tampon pledget relative to the source of bleeding is typically to high, menstrual blood generally contacts one side of the pledget first, typically close to the withdrawal end of the pledget. Conventional pledgets do not absorb and expand sufficiently rapidly to prevent fluid leakage. Moreover, the typical high placement leads to non-uniform absorbency, which may contribute to bypass leakage. While it is preferable that women insert and position tampon pledget lower in the vaginal cavity (e.g., underneath the cervical os) to promote more uniform absorbency, it is believed that discomfort is experienced by the wearer when the tampon is located closer to the introitus. For example, conventional tampon pledgets may exert greater force on the walls of the vagina near the introitus, as the introitus is of a smaller diameter. Also, nerve endings are present in greater number near the introitus and thus may be irritated by the pressure exerted from the tampon. Finally, near the introitus the vaginas of many women are observed to exhibit a sort of “dog-bone” geometry: narrow in the middle and wider at the ends. Insertion of the tampon in this narrow, middle section can be uncomfortable. This anatomical feature, too, no doubt encourages women to place the tampon up one side, usually to the right, anterior side, as we have noted above.

As noted above, tampon pledget placement within the vaginal cavity can contribute to poor leakage performance. While conventional tampon pledgets are highly absorbent, menstrual fluid can bypass a significant portion of the tampon pledget, causing bypass leakage with only a fraction of the potential tampon absorbency being utilized. For example, FIGS. 1A and 1B depict tampons 10 and 20 used by subject females where discoloration, shown generally at 32 and 34, illustrates absorption of menses. As illustrated in FIGS. 1A and 1B, while there is some absorption near the top insertion ends 12 and 22 of the tampons 10 and 20, respectively, absorption is clearly seen at one side (e.g., the side closest to the source of bleeding) and, most notably, at withdrawal ends 14 and 24 of the tampons 10 and 20, respectively. FIGS. 1A and 1B also illustrate little fluid absorption on opposite sides 16 and 26 of the tampons 10 and 20. The inventors recognized that even though conventional tampons are highly absorbent, menstrual fluid typically bypasses a significant portion of the tampon resulting in leakage and only a fraction of the potential absorbency of the tampon being attained.

The inventors conducted investigations to confirm that the pattern of absorbency seen in FIGS. 1A and 1B stemmed mainly from placement of tampon pledgets within the vaginal cavity. FIGS. 2A and 2B depict imaging data, e.g., MRI scans, of subject females that have inserted tampons prior to scans. As shown in FIGS. 2A and 2B, women tend to insert tampons into one of the vaginal fornices, for example, to the anterior and to the right of the cervical os. In one investigation, for example, twelve out of fifteen women scanned inserted tampons in this manner.

Moreover, the inventors have discovered that application of image analysis software to MRI data provides a means to examine an anatomical structure of the female vaginal cavity interacting with a feminine hygiene product, such as a tampon, inserted therein. Modeling and analyzing the interaction of the anatomical structure and the product allows the inventors and like product designers a realistic view of the internal workings of the body. For example, FIGS. 3A-3C illustrate a representation of the vaginal cavity 30 and a tampon 40 disposed therein. As shown in FIGS. 3A-3C, the tampon 40 does not completely fill the vaginal cavity 30 and, thus, channels 50 are formed by which fluid (e.g., menses) could bypass the tampon pledget 40. Since the vaginal cavity has greater elasticity than conventional tampon pledgets, the pledget 40 tends to open up or stretch the width of the vaginal cavity 30 contributing to the formation of bypass leakage channels such as the channels 50. The inventors have determined through analysis that channels are larger when the tampon is placed higher up in the vaginal cavity, for example, within the vaginal fornices as shown in FIGS. 2A and 2B.

Accordingly, it has been discovered that there is a need for an improved tampon pledget design and, in particular, for a tampon pledget that expands rapidly and has greater absorbency near the withdrawal end of the tampon.

### SUMMARY OF THE INVENTION

The present invention is directed to a feminine hygiene product. The feminine hygiene product includes a body having a mass of compressed fibrous material, an insertion end and a withdrawal end. The body has an overall length L. The insertion end has a diameter A and the withdrawal end has a diameter B. In an initial state, diameter B is at least one of equal to up to about ten percent greater than the diameter A. When exposed to a fluid, diameter B radially expands such that a ratio of diameter B to diameter A is about 1.25 to 1.

In one embodiment, the diameter A is a smallest diameter of the body within an upper five percent (5%) of the overall length L. In another embodiment, the diameter A is a smallest diameter of the body within an upper fifty percent (50%) of the overall length L.

In one embodiment, the diameter B is a widest diameter of the body within a lower fifty percent (50%) of the overall length L. In one embodiment, the ratio of the diameter B to the diameter A is in a range of about 1.1-3.0 to 1.

In yet another embodiment, the insertion end and the withdrawal end are formed of different fibrous materials. In one embodiment, the different fibrous materials are comprised of at least one of materials having a different fiber composition, a different web density, a different fiber denier, a different fiber finish and/or are formed using a different method of binding together fibers in a non-woven web. In still another embodiment, the fibrous material at the withdrawal end of the body is comprised of a material having at least one of a faster absorbency rate and a larger absorbency capacity than the material of the insertion end of the body.

### BRIEF DESCRIPTION OF THE DRAWINGS

The features and advantages of the present invention will be better understood when the Detailed Description of the Preferred Embodiments given below is considered in conjunction with the figures provided.

FIGS. 1A and 1B depict tampons used by subject females where discoloration illustrates absorption of menses.

FIGS. 2A and 2B depict imaging data of subject females that have inserted tampons prior to scans.

FIGS. 3A-3C illustrate a representation of the vaginal cavity and a tampon disposed therein.

FIG. 4 illustrates a tampon pledget, configured and operating in accordance with the present invention, prior to use.

FIG. 5 illustrates the tampon pledget of FIG. 4 after use, in accordance with one embodiment of the present invention.

FIG. 6 illustrates the tampon pledget of FIG. 4 after use, in accordance with another embodiment of the present invention.

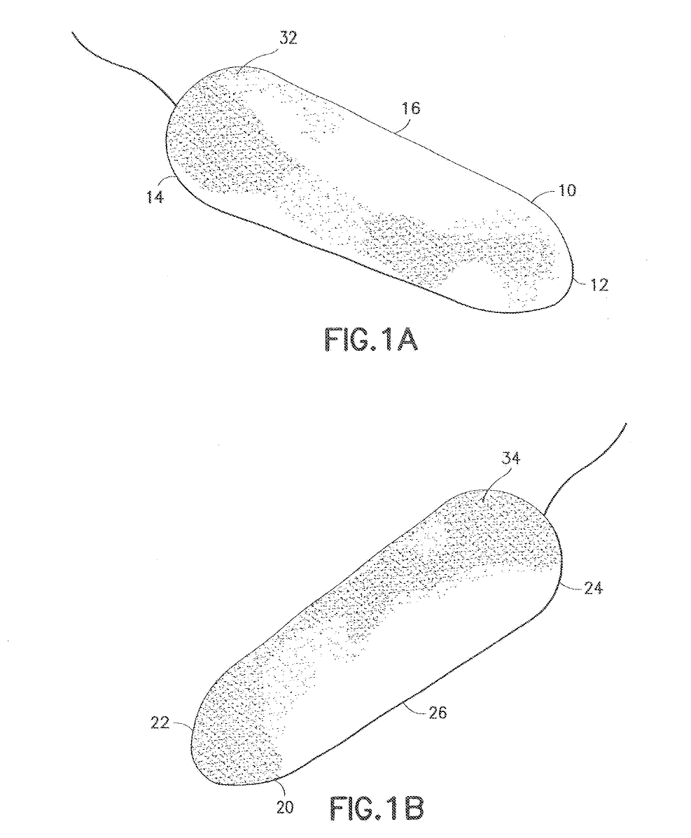

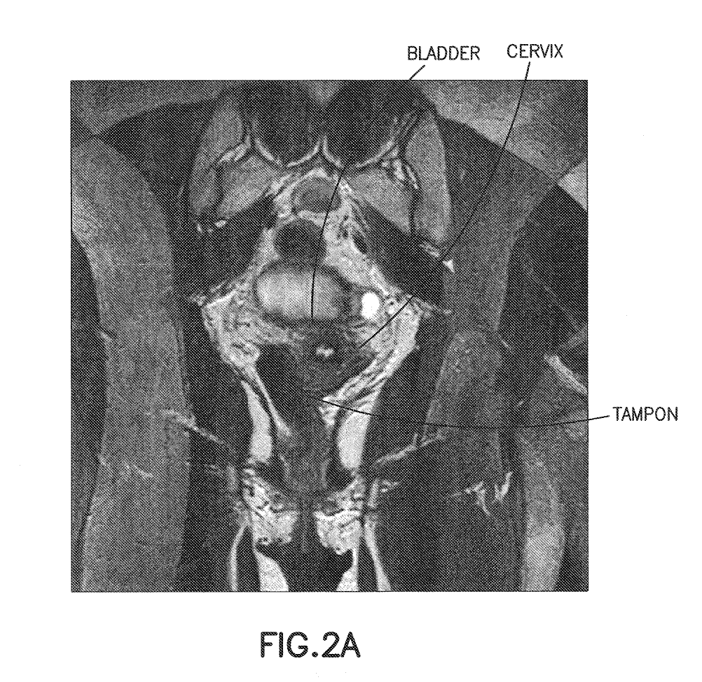

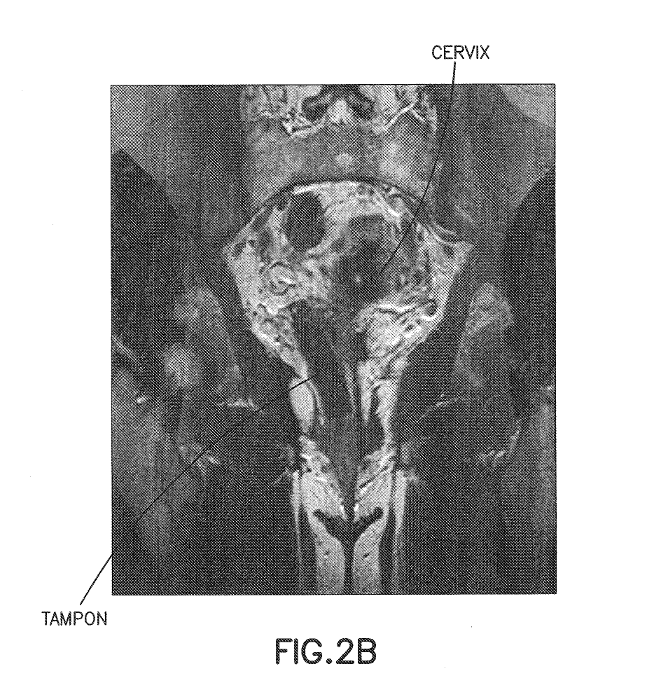

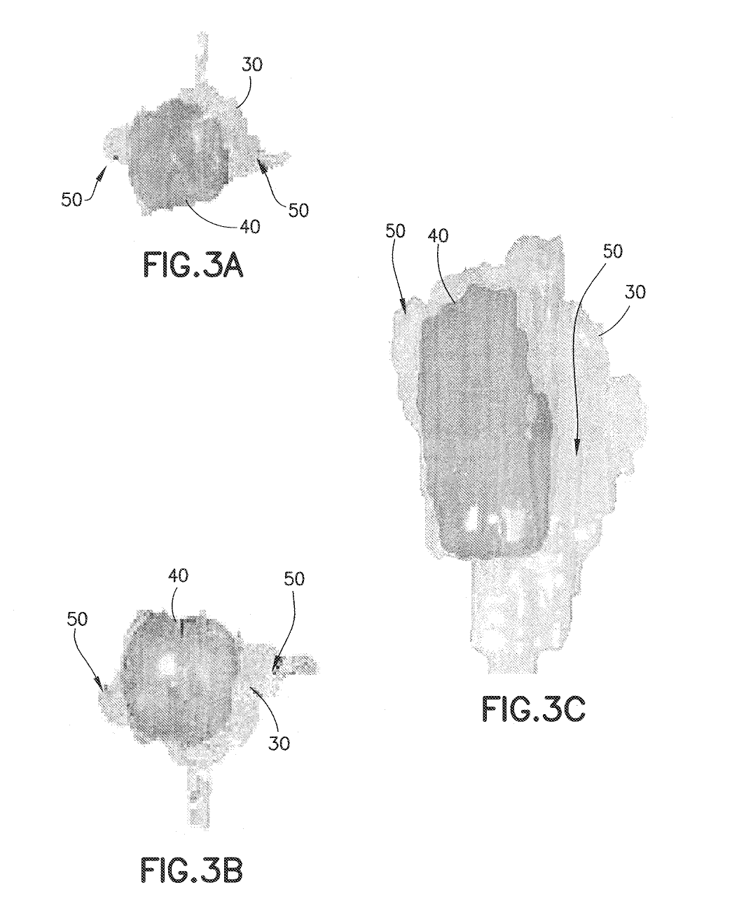

In these figures like structures are assigned like reference numerals, but may not be referenced in the description of all figures.

### DETAILED DESCRIPTION OF THE PREFERRED EMBODIMENTS

The inventors discovered that a pledget with increased absorption and/or expansion properties near the withdrawal end of the pledget is beneficial for leakage protection. The inventors also discovered that an essentially cylindrical tampon pledget provides comfort that consumers prefer.

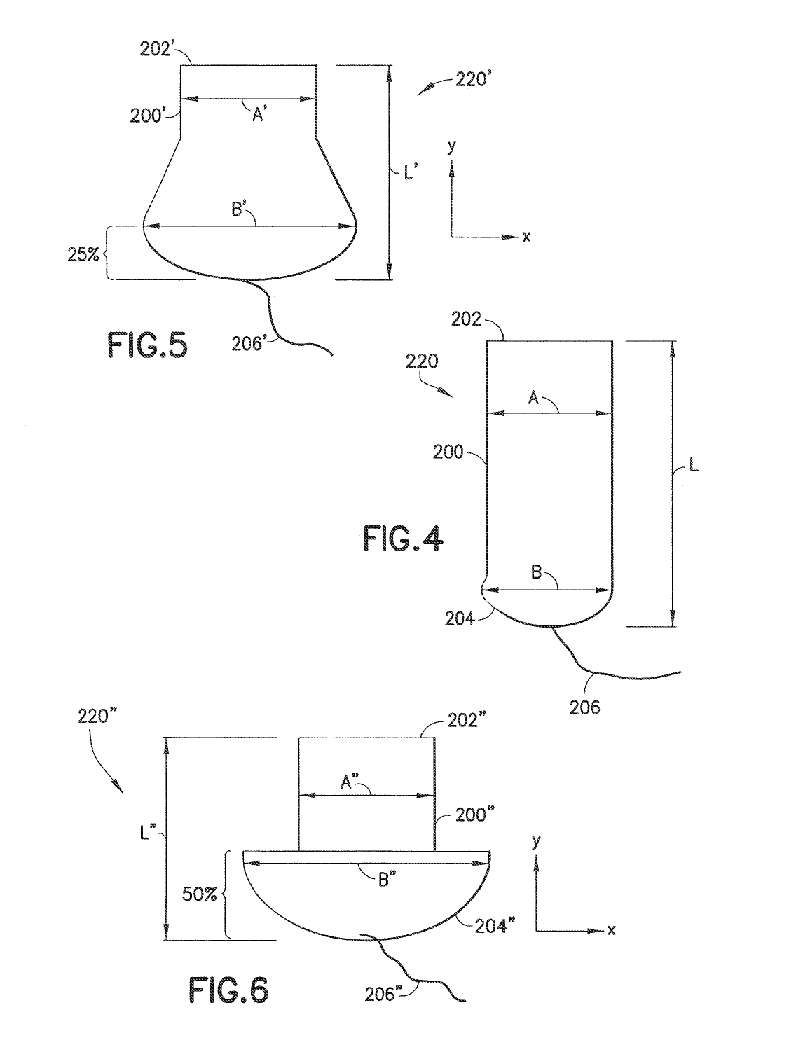

FIG. 4 illustrates a tampon pledget 200 prior to use. The tampon pledget 200 is comprised of a mass of compressed fibrous material having an insertion end 202, a withdrawal end 204 and a string 206 coupled to the withdrawal end. As shown in FIG. 4, the tampon pledget 200 is cylindrical or columnar in shape over its total length L, shown generally at 220. The insertion end 202 has a diameter A. The withdrawal end 204 has a diameter B. In one embodiment, prior to use and expansion, the diameter B is at least equal to or about ten percent (10%) greater that the diameter A. In one embodiment, diameter A is about 13.9 mm and diameter B is about 15.3 mm.

FIG. 5 illustrates one embodiment of the tampon pledget 200 after radial expansion, depicted in the expanded form as a tampon pledget 200′. As shown in FIG. 5, a withdrawal end 204′ of the tampon 200′ rapidly expands due to earlier fluid absorption such that the withdrawal end 204′ seals or plugs the vaginal cavity to substantially prevent early bypass leakage. As shown in FIG. 5, a diameter A′ is in the top five percent (5%) of the length L 220′ of the tampon pledget 200′ (e.g., at an insertion end 202′). A diameter B′ is taken at a widest part of the withdrawal end 204′ of the pledget 200′. In one embodiment, the withdrawal end 204′ is the bottom twenty-five percent (25%) of the length L 220′ of the tampon pledget 200′. In one embodiment, the ratio of diameters B′:A′ is about 1.25:1. In one embodiment, the diameter A′ is a smallest diameter in the topmost fifty percent (50%) of the length L 220′ of the tampon pledget 200′, while the diameter B′ is the widest measurement in the bottom fifty percent (50%) of the length L 220′ of the tampon pledget 200′. In this embodiment, the ratio of diameters B′:A′ is in a range from about 1.1-3.0:1.

FIG. 6 illustrates one embodiment of the tampon pledget 200 after radial expansion, depicted in the expanded form as tampon pledget 200″. As shown in FIG. 6, a withdrawal end 204″ of the tampon 200″ rapidly expanded due to fluid absorption. As shown in FIG. 6, a diameter A″ is taken to be the diameter within the upper five percent (5%) of the length L 220″ of the tampon pledget 200″ and a diameter B″ is the diameter taken at a widest part of a bottom fifty percent (50%) of the length 220″ of the tampon pledget 200″. In this embodiment, the ratio of the diameters B″:A″ is about 1.25:1. In yet another embodiment, the diameter A″ is taken as a smallest diameter in the top fifty percent (50%) of the length L 220″ of the pledget 200″, while the diameter B″ is a widest measurement in a bottom fifty percent (50%) of the length 220″ of the pledget 200″ and the ratio B″:A″ is in a range from between 1.1-3.0:1.

As described above, the diameters B, B′ and B″ (hereinafter referred to collectively as diameter B) increase due to early fluid absorption in the wet environment of the female vagina, which results in the tampon sealing or plugging the vaginal cavity sufficiently and quickly enough to prevent early bypass leakage. In one embodiment, the diameter B expansion is due both to an absorbency rate as well as an absorbent capacity. That is, the withdrawal end 204 of the tampon pledget (e.g., the diameter B portion) is comprised of material having a faster absorbency rate and, in one embodiment, also having a larger absorbent capacity then material comprising the insertion end 202 of the tampon pledget 200 (e.g., the diameter A portion). As illustrated in FIGS. 5 and 6, in one embodiment, the tampon pledget is comprised of a cylindrical or elliptical cross-sectional shape. However, it should be appreciated that it is also within the scope of the present invention for the configuration of the tampon pledget 200 to include a slight taper from a broader withdrawal end to a narrower insertion end. In one embodiment, the configuration may be curved to, for example, accommodate the natural curve of the female vagina. In general, an overall shape of an applicator containing tampon pledgets of the present invention is cylindrical or elliptical.

The inventors have discovered that to achieve the rapid expansion and formation of a sealing shape (e.g., achieve the diameter B) near the withdrawal end, changes in fibrous materials, non-woven webs, web manufacture, and/or tampon forming methods are employed. For example, the withdrawal end 204 of the pledget 200 is less compressed than the middle and insertion end 202 of the pledget 200. In one embodiment, different compression techniques are employed when forming the insertion versus the withdrawal ends of the pledget. For example, a special crimping tool or a special two-part oven tube is used that is designed to provide more compression in the top two-thirds of the pledget (e.g., at the insertion end) than in the bottom third (e.g., at the withdrawal end). Moreover, a crimping tool or oven tube is tapered or splined to provide an aesthetically pleasing shape. It is also within the scope of the present invention to apply heat and/or pressure differentially when forming the insertion versus the withdrawal ends of the tampon.

For example, an oven tube having an inner diameter that increases from an inside diameter of, say, 13 mm near the top and middle to an inside diameter of, say, 15 mm near the bottom or withdrawal end could be used to make pledgets of this invention. Furthermore, by applying slightly less infrared heating to the bottom or withdrawal end as opposed to that applied to the middle and top portions, one could get the pledget to take a compression set more near the top and encourage the pledget to expand more rapidly and to a greater extent near the withdrawal end. Such differential expansion should help plug up the bottom portion of the vaginal vault and thus reduce bypass leakage. One way to implement this would be to use heating elements to heat up the oven tube to, say, 270 deg F. near the top and middle portions, but keeping the bottom portion cooler, say, by heating only to about 210 deg F.

In one embodiment, the insertion end 202 is formed of different fibers or fibrous web than are used at the withdrawal end 204. For example, at the withdrawal end 204 of the pledget 200, a higher denier, rayon/PET fibrous web provides rapid wicking and, thus, more rapid expansion as compared to a lower denier cotton web, which the inventors have found to be more suitable for the insertion end 202 of the pledget 200. Several non-woven web pieces together provide greater thickness and/or a higher diameter in the bottom third of the pledget (e.g., at the withdrawal end). It should be appreciated that by changing the fiber composition, by changing the web density, by changing the method of binding together fibers in the non-woven web, and/or by changing the fiber denier or fiber finish, the pledget 200 responds to fluid intake more quickly in one portion of the pledget, e.g., at the withdrawal end 204 (e.g., the bottom third of the length), than in the insertion end 202 (e.g., the top two-thirds of the length) of the tampon pledget 200.

It should be appreciated that the present invention also incorporates design principals that are used in conventional tampon pledget designs. For example, the inventive tampon pledgets may be a digital or applicator-type tampons. Moreover, the inventive tampon pledgets include absorbent fibers such as, for example, rayon, cotton, cellulosic fluff, pulp, and the like, as the primary absorbent means. It is also within the scope of the present invention to include super-absorbent and fiber blends involving non-absorbent fibers. Also, tampons of the present invention may optionally include overwrap and/or coverstock, both to prevent fiber sloughing as well as to provide a smooth, comfortable feel. In one embodiment, strings 206 coupled to withdrawal end 204 include a coated cotton fiber.

The inventors have found a number of useful methods for making tampon pledgets of the present invention. For example, the inventors have discovered that methods for making tampon pledgets configured and operating in accordance with the present invention may be folded, radially compressed, cross-pad, non-woven based pledgets using methods similar to those employed to make tampons sold by the assignee of the present invention within its GENTLE GLIDE® brand tampon pledgets. While the manufacturing methods may be similar, when making the inventive tampon pledgets a number and/or dimensions of absorbent pads used prior to compression are varied. For example, pads of the present invention are selected such that when the tampon pledget is formed by folding and subsequent compression, the pledget includes a slightly larger percentage of mass (e.g., higher absorbency) in the withdrawal end of the pledget. In one embodiment, a dimension that is changed within the inventive pledget design as compared to convention design is at least one of the length, width and thickness of a web piece used to produce the two crosspads. In one embodiment, a different density is used for the two web pieces to influence the absorbency and/or the absorbency rate. This is done by both or either using fibers exhibiting different absorbencies and/or by changing the methods of bonding or compressing the non-woven webs used in the pads. For example, if the web is bonded together using needle punching, a higher concentration of needles could be used to influence the web density.

It should be appreciated that it is within the scope of the present invention for the manufacturing parameters outlined above, e.g., use of a different number of pads, pad lengths, pad widths, pad thicknesses, pad basis weights, and fiber materials and blend compositions used to make up the pads, to change together or individually. Table 1 illustrates exemplary pad configurations used to manufacture a tampon pledget in accordance with one embodiment of the present invention.

TABLE 1
Exemplary Tampon Pledget Configuration

| | Pad | | Pad | Pad | Total |
|---|---|---|---|---|---|
| | Configura- | Pad Weight | Length, | Width, | Target |
| Example | tion | (targeted), g | in. | in. | Weight, g |
| Comparative | Inner | 1.725 | 4.0 | 2.375 | |
| Comparative | Outer | 1.725 | 2.375 | 4.0 | 3.45 |
| 1 | Inner | 2.179 | 4.0 | 3.0 | |
| 1 | Outer | 1.271 | 3.125 | 2.240 | 3.45 |

As illustrated in Table 1, variation is seen in pad lengths and widths for the two pads (e.g., inner and outer) used to make the tampon pledget configurations. The dimensional changes produce pad weight changes but, as is shown in Table 1, the total weight of both pads remain the same, e.g., 3.45 grams. In manufacturing the pledgets identified in Table 1, the same fiber (e.g., a Galaxy® brand multilobal rayon fiber, available from Kelheim Fibres GmbH, of Kelheim, Germany) was used to make the web pieces that make up these pads. Also the web basis weights were substantially the same. The tampon pledgets were folded and formed using an HP simulator machine available from, for example, Hauni Richmond, Inc., Richmond, Va., USA. Oven tubes used to form the cylindrical pledgets, and temperatures and times for heating and compressing the pledgets were nearly identical for these two sets of tampons. In one test scenario, approximately forty (40) tampons were made for each of the configurations listed in Table 1. In one embodiment, GENTLE GLIDE™ brand applicators were used to assemble applicator tampons.

The inventors performed testing to evaluate the tampon pledget configurations illustrated in Table 1. A cutting device such as, for example, a deli slicer (e.g., a Chef's Choice International Electric Food Slicer Model #662, available from Edgecraft Corporation, Avondale, Pa., USA), was used to cut each of the tampon configurations along an axis running lengthwise through the tampon. Once cut, the tampon was examined to determine how the weight was distributed for the two configurations of tampons of Table 1. Several slices were then grouped together to form four “quartiles”. Because the deli slicer cuts almost perfectly uniform slices, these quartiles are of nearly equal length along the longitudinal pledget axis. The slice weights were determined and percentages for each quartile were determined for these two sets of tampons. Table 2 provides weight distribution comparisons. Within Table 2, quartile 1 is closest to the withdrawal or string end.

TABLE 2
Weight Distributions for Tampons of Table 1.

| Quartile | Comparative | Example 1 |
|---|---|---|
| 1 | 7.73% | 10.33% |
| 2 | 30.24% | 26.34% |
| 3 | 36.05% | 32.14% |
| 4 | 25.98% | 17.22% |

As is shown in Table 2, Example 1 exhibits a greater percentage of mass near the string or withdrawal end (Quartile 1) versus the Comparative Example (e.g., 10.33% as compared to 7.73%). While the percentage of mass is not a large difference, the inventors have discovered that the percentage of mass is adjusted by other types of pledget changes suggested above. For example, variations in pad thickness and fiber materials in addition to the variations in pad length and widths illustrated in Tables 1 and 2.

The inventors conducted further tests, including syngyna absorbencies, to determine absorbency characteristics for the two configurations of tampon pledgets of Table 1. The results are provided in Table 3.

TABLE 3
Syngyna Absorbency Data for Configurations of Table 1.

| | | | | |
|---|---|---|---|---|
| Sample | Dry Wt. | Wet Wt. | Absorbency | g per g absorbency |

Comparative Example
Syngyna: 10 Tampons Tested. 5 tested for Moisture. Corrections
Applied to Compute gram per gram absorbency

| | | | | |
|---|---|---|---|---|
| 1 | 3.39 | 17.45 | 14.06 | 5.06 |
| 2 | 3.63 | 19.21 | 15.58 | 5.20 |
| 3 | 3.27 | 17.82 | 14.55 | 5.34 |
| 4 | 3.32 | 17.84 | 14.51 | 5.27 |
| 5 | 3.16 | 17.36 | 14.20 | 5.39 |
| 6 | 3.40 | 17.43 | 14.03 | 5.04 |
| 7 | 3.39 | 17.85 | 14.46 | 5.17 |
| 8 | 3.23 | 17.31 | 14.07 | 5.26 |
| 9 | 3.15 | 16.97 | 13.82 | 5.29 |
| 10 | 3.37 | 17.33 | 13.96 | 5.05 |
| Average | 3.51 | 18.33 | 14.82 | 5.13 |
| Std Dev | 0.14 | 0.61 | 0.51 | 0.13 |
| High | 3.63 | 19.21 | 15.58 | 5.39 |
| Low | 3.15 | 16.97 | 13.82 | 5.04 |

Example 1. Same Number tested for Syngyna Absorbency and
Moisture Levels and Same Corrections Applied.

| | | | | |
|---|---|---|---|---|
| 1 | 3.43 | 18.21 | 14.78 | 5.09 |
| 2 | 3.56 | 19.65 | 16.09 | 5.29 |
| 3 | 3.43 | 18.44 | 15.01 | 5.15 |
| 4 | 3.51 | 18.10 | 14.59 | 4.94 |
| 5 | 3.35 | 17.76 | 14.41 | 5.08 |
| 6 | 3.68 | 19.97 | 16.30 | 5.20 |
| 7 | 3.57 | 18.73 | 15.16 | 5.03 |
| 8 | 3.39 | 18.59 | 15.20 | 5.26 |
| 9 | 3.37 | 17.99 | 14.62 | 5.12 |
| 10 | 3.45 | 18.22 | 14.78 | 5.07 |
| Average | 3.49 | 18.93 | 15.44 | 5.19 |
| Std Dev | 0.10 | 0.72 | 0.63 | 0.11 |
| High | 3.68 | 19.97 | 16.30 | 5.29 |
| Low | 3.35 | 17.76 | 14.41 | 4.94 |

P value for t test comparing above examples
(significantly different if <0.05)

| | | | | |
|---|---|---|---|---|
| P | 0.020 | 0.007 | 0.008 | 0.128 |

As is illustrated in Table 3, there are slight differences in the pledget weights and the actual weights were higher than a 3.45 gram target. The dry weights, wet weights and absorbencies are slightly different, but the grams per gram absorbencies are 5.13 (comparative) and 5.19 (Example 1), which are comparable and not statistically significantly different (P=0.128 by the t test).

Table 4 below includes comparisons of measured pledget diameters near the string end for the two configurations of tampon pledget of Table 1, illustrating results both for pledgets before expansion by the syngyna test and for those that had expanded during the syngyna absorbency testing. As is illustrated in Table 4, the tampon pledgets of the present invention expanded at the string end, on average, to a greater extent (a 70.7% diameter increase) than those for the comparative example (a 55% diameter increase). When the data for the two pledgets of Table 1 are compared for all five pledgets at both orientations, results for the expanded diameters are statistically significantly different, based on a two-tailed T test at ninety-five percent (95%) confidence, even though the gram per gram absorbencies are not significantly different.

TABLE 4
Expansion Characteristics for Pledgets of Table 1.

| | String End, Before Syngyna | | | STRING END, After Syngyna | | |
|---|---|---|---|---|---|---|
| | First | after 90 deg. | Average of Both | First | after 90 deg. | Average of Both |
| Sample | Measurement | Rotation | Orientations | Measurement | Rotation | Orientations |

Comparative Example. Vision Engineering Hawk Dynascope used to Measure Diameters (Inches).
5 Samples Tested, 2 Orientations.

| | | | | | | |
|---|---|---|---|---|---|---|
| 1 | 0.563 | 0.582 | 0.573 | 0.898 | 0.883 | 0.890 |
| 2 | 0.558 | 0.559 | 0.559 | 1.018 | 1.000 | 1.009 |
| 3 | 0.564 | 0.576 | 0.570 | 0.778 | 0.825 | 0.802 |
| 4 | 0.563 | 0.564 | 0.563 | 0.837 | 0.845 | 0.841 |
| 5 | 0.565 | 0.572 | 0.569 | 0.844 | 0.853 | 0.848 |
| Average | 0.563 | 0.571 | 0.567 | 0.875 | 0.881 | 0.878 |
| Std Dev | 0.003 | 0.009 | 0.006 | 0.090 | 0.070 | 0.080 |
| High | 0.565 | 0.582 | 0.573 | 1.018 | 1.000 | 1.009 |
| Low | 0.558 | 0.559 | 0.559 | 0.778 | 0.825 | 0.802 |
| | | | | | % Increase | 55.0% |
| | | | | | in Diameter | |

Example 1. Vision Engineering Hawk Dynascope used to Measure Diameters (inches).
5 Samples Tested, 2 Orientations.

| | | | | | | |
|---|---|---|---|---|---|---|
| 1 | 0.563 | 0.558 | 0.561 | 0.995 | 0.994 | 0.994 |
| 2 | 0.566 | 0.563 | 0.564 | 0.946 | 0.931 | 0.938 |
| 3 | 0.560 | 0.571 | 0.566 | 0.969 | 0.986 | 0.977 |
| 4 | 0.558 | 0.557 | 0.557 | 0.974 | 0.975 | 0.975 |
| 5 | 0.562 | 0.571 | 0.567 | 0.929 | 0.913 | 0.921 |
| Average | 0.562 | 0.564 | 0.563 | 0.963 | 0.960 | 0.961 |
| Std Dev | 0.003 | 0.007 | 0.004 | 0.026 | 0.036 | 0.030 |
| High | 0.566 | 0.571 | 0.567 | 0.995 | 0.994 | 0.994 |
| Low | 0.558 | 0.557 | 0.557 | 0.929 | 0.913 | 0.921 |
| | | | | | % Increase | 70.7% |
| | | | | | in Diameter | |

The results depicted in Table 4 show that the inventive tampon pledgets expand to a greater extent near the string or withdrawal end, as is desired. Given the in-body, computational and laboratory results provided earlier, the inventors realized that such tampon pledget configurations are preferable from both a comfort and leakage protection standpoint.

Although described in the context of preferred embodiments, it should be realized that a number of modifications to these teachings may occur to one skilled in the art. Accordingly, it will be understood by those skilled in the art that changes in form and details may be made therein without departing from the scope and spirit of the invention.

What is claimed is:

1. A feminine hygiene product, comprising:
a body including a mass of compressed fibrous material having an insertion end and a withdrawal end, the body having an overall length L, the insertion end having a diameter A and the withdrawal end having a diameter B;
wherein when in an initial state, diameter B is at least about equal to up to about ten percent greater than the diameter A, and wherein when exposed to a fluid, diameter B radially expands such that a ratio of diameter B to diameter A is about 1.25 to 1.

2. The feminine hygiene product of claim 1, wherein the diameter A is a smallest diameter of the body within an upper five percent of the overall length L.

3. The feminine hygiene product of claim 1, wherein the diameter B is a widest diameter of the body within a lower fifty percent of the overall length L.

4. The feminine hygiene product of claim 1, wherein the diameter A is a smallest diameter of the body within an upper fifty percent of the overall length L.

5. The feminine hygiene product of claim 4, wherein the diameter B is a widest diameter of the body within a lower fifty percent of the overall length L.

6. The feminine hygiene product of claim 5, wherein the ratio of the diameter B to the diameter A is in a range of about 1.1-3.0 to 1.

7. The feminine hygiene product of claim 1, wherein the insertion end and the withdrawal end are formed of different fibrous materials.

8. The feminine hygiene product of claim 7, wherein the different fibrous materials are comprised of at least one of materials having a different fiber composition, a different web density, a different fiber denier, a different fiber finish and are formed using a different method of binding together fibers in a non-woven web.

9. The feminine hygiene product of claim 8, wherein the different fiber denier includes the withdrawal end of the body having a higher denier fibrous web as compared to a lower denier fibrous web at the insertion end.

10. The feminine hygiene product of claim 8, wherein the different method of binding includes compressing the body such that the withdrawal end of the body is less compressed than the insertion end and any intervening portion of the body.

11. The feminine hygiene product of claim 7, wherein the fibrous material at the withdrawal end of the body is comprised of a material having at least one of a faster absorbency rate and a larger absorbency capacity than the material of the insertion end of the body.

12. A vaginal tampon, comprising:
a body including a mass of absorbent material having an insertion end and a withdrawal end, the body having an overall length L, the insertion end having a diameter A and the withdrawal end having a diameter B;
wherein when exposed to a fluid, diameter B expands more rapidly than diameter A to substantially seal a bottom portion of a vaginal cavity and prevent bypass leakage.

13. The tampon of claim 12, wherein the diameter A is a smallest diameter of the body within an upper five percent of the overall length L.

14. The tampon of claim 12, wherein the diameter B is a widest diameter of the body within a lower fifty percent of the overall length L.

15. The tampon of claim 12, wherein prior to exposure to the fluid diameter B is at least one of equal to and ten percent greater than the diameter A, and after exposure to the fluid, diameter B expands such that a ratio of diameter B to the diameter A is about 1.25 to 1.

16. A process for making a tampon of claim 12, wherein the tampon body is formed of a plurality of pads, each of the plurality of pads having a different length and width dimension, the plurality of pads are formed by folding and compressing such that, in an initial state, diameter B is at least one of equal to and ten percent greater than the diameter A.

17. The process of claim 16, wherein after formation the body includes a larger percentage of mass at the withdrawal end as compared to the insertion end.
